(19) United States
(12) Patent Application Publication
(10) Pub. No.: US 2016/0310080 A1
(43) Pub. Date: Oct. 27, 2016

(54) POLYMER-BASED CARDIOVASCULAR BIOSENSORS, MANUFACTURE, AND USES THEREOF

(71) Applicant: UNIVERSITY OF SOUTHERN CALIFORNIA, LOS ANGELES, CA (US)

(72) Inventors: Tzung K. Hsiai, Santa Monica, CA (US); Hongyu Yu, Tempe, AZ (US); Eun Sok Kim, Rancho Palos Verdes, CA (US); Lisong Ai, Irvine, CA (US)

(73) Assignee: UNIVERSITY OF SOUTHERN CALIFORNIA, LOS ANGELES, CA (US)

(21) Appl. No.: 14/947,480

(22) Filed: Nov. 20, 2015

Related U.S. Application Data
(63) Continuation of application No. 12/134,938, filed on Jun. 6, 2008, now abandoned.
(60) Provisional application No. 60/942,300, filed on Jun. 6, 2007.

(51) Int. Cl. A61B 5/00; B05D 7/00; A61B 5/0215; A61B 5/021; A61B 5/01; A61B 5/027

(52) U.S. Cl. CPC A61B 5/6852; A61B 5/01; A61B 5/027; A61B 5/02156; A61B 5/02141; B05D 7/50; A61B 2562/0261; A61B 2562/0271; A61B 2562/125; A61B 2562/222

(57) ABSTRACT

A flexible, polymer-based biosensor deployable into the arterial system which can assess shear stress in the arterial geometry in the presence of time-varying component of blood flow. Also, a method of fabricating a biosensor which may be used for in vivo procedures, involving the sequential depositing onto a substrate of a silicon dioxide layer, a metal heating element on the silicon dioxide layer, and a biocompatible polymer on the heating element, followed by etching the polymer layer to provide holes to allow for electrode contact with the heating element. A second metal layer is then deposited to form electrodes, followed by a second biocompatible polymer layer to form the device structure and removing the fabricated biosensor from the substrate by etching the substrate. In addition, a method of determining intravascular shear stress by measuring the temperature, flow rate and pressure of a bodily fluid with a biocompatible biosensor is disclosed.

### CROSS REFERENCE TO RELATED APPLICATIONS

This application is a continuation of U.S. patent application Ser. No. 12/134,938, filed Jun. 6, 2008, entitled “Polymer-Based Cardiovascular Biosensors, Manufacture and Uses Thereof,” attorney docket 028080-0350; which is based upon and claims the benefit of priority from Provisional U.S. Patent Application 60/942,300, filed Jun. 6, 2007, attorney docket 028080-0276. The entire contents of both applications are incorporated by reference herein.

### STATEMENT REGARDING FEDERALLY SPONSORED RESEARCH

This invention was made with government support under Contract Nos. HL083015 and HL068689 awarded by the National Institutes of Health. The government has certain rights in the invention.

### BACKGROUND

1. Field of the Disclosure

This disclosure resides in the field of biosensors. Specifically, the disclosure is directed to a polymer-based biosensor able to be used in conjunction with a catheter for in vivo analysis of body fluid temperature, pressure, flow rate and fluid shear stress. The disclosure is also directed towards the manufacture of a polymer-based biosensor and the uses thereof.

2. Description of the Related Art

Coronary artery disease remains the leading cause of death in the United States and is an emergent global health issue. Hemodynamic forces, specifically, fluid shear stress, play an important role in the biological activities of cardiovascular endothelial cells. Evidence shows that variations in flow velocity, low wall shear stress, flow separation, and turbulence favor the pathogenesis of arteriosclerosis. The characteristics of shear stress have been implicated in a variety of vascular responses from angiogenesis, vascular permeability, inflammatory responses, as well as activation of mitogenic, thrombogenic and fibrinolytic factors to recruitment of inflammatory cells at the microcirculation level.

At arterial bifurcations where inflammatory processes prevail, the fluid mechanical environment is distinct from the laminar pulsatile environment present in the long and straight regions of the vessel or the medial wall within bifurcations. At the lateral walls of arterial bifurcations, disturbed flow, including oscillatory flow (bidirectional net zero forward flow), is considered to be an inducer of vascular oxidative shear stress that promotes the initiation and progression of atherosclerosis.

Measuring the vessel wall shear stress precisely remains as a challenging issue, although several methods have been developed for wall shear stress measurement by non-direct methods. For example, one non-direct method is optical velocimetry, which uses a laser Doppler velocimeter or a particle image velocimeter. However, this method results in excessive noise generated in the signal due to the reflection from the wall.

One direct method of measuring shear stress called thermal anemometry. The operation principle is based on convective cooling of a heated sensing element as fluid flows over its surface. The heat transfer from the heated surface to the fluid depends on the flow characteristics in the viscous region of the boundary layer. When an electric current passes through the heated element, the heat convection from a resistively heated element to the flowing fluid is measured. From this, the value of shear stress may be inferred. The advantages of this technique are simplicity in fabrication, absence of moving elements, and good sensitivity. Thus, this method provides a basis to develop micro intravascular sensors on a single silicon wafer for high throughput production.

Micro electro mechanical systems (MEMS) technology explores the science of the micro realm, in which the surface tension and viscous force, rather than the force of gravity, influence the design and operation of sensors and devices. MEMS shear stress sensors have been developed for aerodynamics and fluid mechanics. Previously, MEMS shear stress sensors have been fabricated with backside wire bonding to address micro-scale hemodynamics with high temporal and spatial resolution. However, current MEMS sensors are relatively inflexible, and unable to be utilized inside a living organism without undue injury to the tissues.

### SUMMARY

In order to overcome the above mentioned problems, this disclosure identifies a flexible, micro polymer-based biosensor which is deployable into the arterial system and can assess shear stress in the complicated arterial geometry in the presence of time-varying component of blood flow.

The disclosure also identifies a novel method of fabricating a biosensor which may be used for in vivo procedures. The method involves the steps of the sequential depositing onto a substrate of a silicon dioxide layer, a metal heating element on the silicon dioxide layer, and a biocompatible polymer on the heating element. The biocompatible polymer is then etched to provide holes to allow for electrode contact with the heating element. Then, a second metal layer is deposited to form electrodes, followed by a second biocompatible polymer layer to form the device structure. In addition, the method may also include a step of removing the fabricated biosensor from the substrate by etching the substrate.

This disclosure also identifies a method of determining intravascular shear stress by measuring the temperature of a bodily fluid with a biocompatible biosensor. The method involves the steps of attaching the biosensor to the terminal end of a coaxial wire capable of measuring electrical resistance in a living organism, inserting the catheter into a living organism (blood vessels) as a conduit, cannulating the coaxial wire with the sensor through the catheter into the bodily fluid (blood) such that the biosensor contacts the bodily fluid and then determining the temperature of the bodily fluid by converting the electrical resistance measured into temperature based on a coefficient of resistance of the biosensor.

In addition to determining the temperature of the bodily fluid, the biosensor may also determine the flow rate of the bodily fluid by calibrating the resistance measurement with flow rate or determine the pressure of the bodily fluid by calibrating the resistance measurement with pressure.

### BRIEF DESCRIPTION OF THE DRAWINGS

FIG. 1A is a view of the biosensor, the sensing element and the electrodes in accordance with one embodiment of the present disclosure.

FIG. 1B and FIG. 1C are a cross-sectional view and side view of the flexible intravascular sensor in accordance with one embodiment of the present disclosure.

### DETAILED DESCRIPTION OF ILLUSTRATIVE EMBODIMENTS

The present disclosure describes a polymer based cardiovascular biosensor. In one preferred embodiment, the biosensor comprises a sensing element; a first and a second metal electrode both of which are in contact with the sensing element; and a biocompatible polymer layer encompassing the first and second electrodes. The use of a biocompatible polymer allows for the in vivo diagnosis of cardiovascular disease.

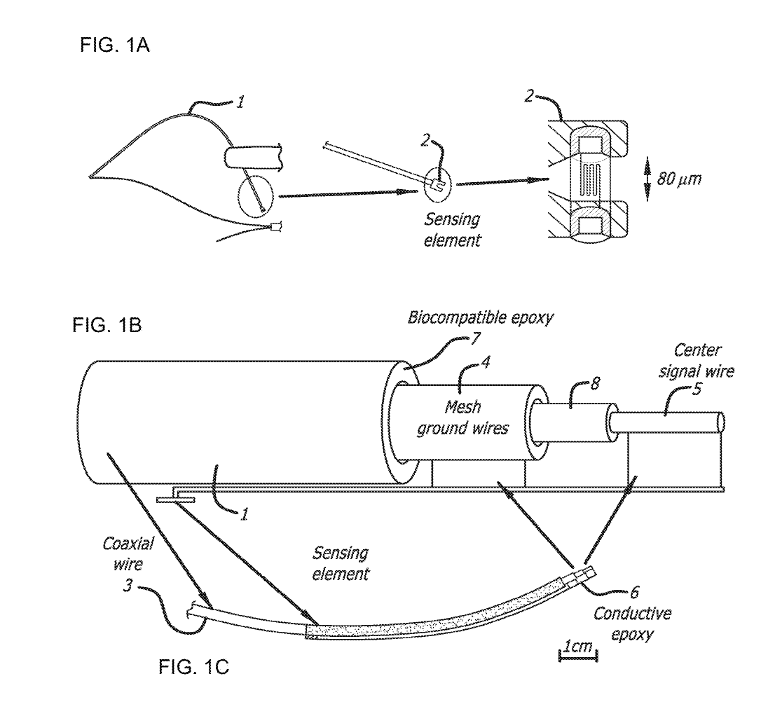

As is shown in FIG. 1A, the biosensor 1 may be bended or folded without structural or functional damage. The sensing element 2 is positioned at the tip of the sensor. In the example shown in FIG. 1A, the sensing element 2 was made of 2 μm wide Ti/Pt strip with a dimension of 240 μm×80 μm. However, any biocompatible electrode material capable of being machined for use as an electrode may be used.

FIG. 1B shows the sensing element 1 at the terminal end of the biosensor attached to the electrical coaxial wire 3 with conductive epoxy 6 and covered with biocompatible plastic resin 7 to prevent from electrical current leakage. Generally, any biocompatible polymer may be utilized to cover the coaxial wire 3. The biocompatible plastic resin layer is preferably comprised of at least one from the group of poly p-chloroxylylene, polyamide, polyimide, polyurethane, and epoxide resin. More preferably, the biocompatible plastic resin layer is comprised of poly-p-chloroxylylene.

Poly-p-chloroxylylene is also known commercially at Parylene C®. Parylene C® is a polymer derived from the monomer chloro-p-xylene, with a molecular weight generally about 500,000 daltons. One feature of Parylene C® is that is may be formed in extremely thin layers. The voltage withstanding properties of Paraylene C® are excellent, and Parylene C® also exhibits excellent thermal, cryogenic, chemical and impact resistance.

In another preferred embodiment, the biosensor further comprises a center signal wire 5 in contact with the first electrode; an insulating layer 8 encompassing the periphery of the center signal wire; a metal ground 4 in contact with the second electrode and encompassing the periphery of the insulating layer; and a biocompatible polymer layer 7 encompassing the periphery of the metal ground. For example, FIG. 1C shows the above mentioned arrangement. The distance between the sensing element and the tip of the catheter may be 4 cm which was designed based on entrance length to avoid flow disturbance. The packaged sensor 1 on electrical coaxial wire 3 are connected. The length of the biosensor may be adjusted according to the needs of the user. The flexibility of the device allows for the user to perform measurement operations inside the small dimensions of arteries.

Preferably, the sensing element is attached to the center signal wire with a conductive biocompatible polymer. More preferably, the sensing element is further attached to the metal ground with conductive biocompatible polymer. Also, preferably, the conductive biocompatible polymer is comprised of conductive epoxy resin, although any suitable biocompatible resin known in the art may be used.

The present disclosure also describes a method of manufacturing a biosensor comprising the steps of depositing a silicon oxide layer on a substrate; depositing and patterning a first metal sensor on the silicon oxide layer; depositing a first plastic resin layer on the metal sensor; etching at least two through holes in the first plastic resin layer; depositing a second metal layer on the plastic resin layer such that a portion of the second metal layer contacts the first metal layer and a portion of the second metal layer contacts the plastic resin layer; and depositing a second plastic resin layer over the second metal layer.

The deposition of the silicon oxide, the metal sensors and the biocompatible polymer may be performed by any known method to those skilled in the art. For example, dry thermal growth, E-beam evaporation, vapor phase deposition, vacuum coating are preferred methods. More preferred methods are E-beam evaporation and dry thermal growth. An example of vapor phase deposition of the biocompatible polymer involves vaporization of the dimer at approximately 175° C. in a vapor deposition chamber. The temperature is then heated to 690° C. to form a stable monomeric diradical of p-chloroxylene. Then the monomer is transferred to a room temperature deposition chamber in which it adsorbs and polymerizes. In another preferred embodiment, Silicon on insulator (SOI) substrates are purchased with the silicon dioxide predeposited on a silicon substrate, thereby foregoing the need for a separate step of depositing the silicon dioxide.

Preferably, the method of fabricating the biosensor may include a step of separating the substrate from the silicon oxide layer. This method is useful in that the biosensor can be removed from the dispensable substrate by etching the substrate from the biosensor.

The metal layers may be comprised of any biocompatible metal capable of conducting a current and exhibiting stability under in vivo conditions. In one preferred embodiment, the first metal layer is comprised of Pt and Ti and the second metal layer is comprised of Au and Cr. Moreover, the second metal layer may be in direct contact with the first metal layer.

The metal sensor is preferably structured to conform to various anatomic curvatures. In addition, the sensor preferably has excellent mechanical strength. A major portion of the sensor is encapsulated in a biocompatible polymer to provide flexible electrical connection in combination with a catheter to transmit electric signals to an external detection circuit. The sensor may further comprise a heating element.

This disclosure also describes uses of the polymer-based biosensor. One use of the biosensor is a method of measuring the temperature of bodily fluid comprising the steps of equipping the inner portion of a catheter with a biosensor capable of measuring electrical resistance inside a living organism; inserting the catheter into a living organism; inducing a bodily fluid to flow into the catheter such that the biosensor contacts the bodily fluid; and determining the temperature of the bodily fluid by converting the electrical resistance measured into temperature based on a coefficient of resistance of the biosensor.

In another preferred method, the biosensor is attached to the terminal end of a coaxial wire with a biocompatible polymer insulating layer. In addition, the method can also include in the step cannulating the coaxial wire with the biosensor through the catheter to allow the biosensor to contact the bodily fluid.

Other uses of the biosensor that may be used with this method include determining the flow rate of the bodily fluid by calibrating the resistance measurement with flow rate and determining the pressure of the bodily fluid by calibrating the resistance measurement with pressure. Of course, these uses are not meant to be limiting in scope, as the biosensor has a variety of other uses in addition to those described herein.

### EXAMPLES

The following examples are offered for purposes of illustration and are not intended to limit the scope of the invention.

### Example 1

Below is one example of the manufacture of a biosensor according to the present disclosure.

The sensor was fabricated using surface micromachining with biocompatible materials including Parylene C, Ti and Pt. To dovetail to the arterial circulation, the sensors were fabricated with (1) dry thermal growth of 0.3 μm SiO₂ and deposition of a 1 pm sacrificial silicon layer using E-beam evaporator, (2) deposition and patterning Ti/Pt layers with thickness of 0.035 μm/0.060 μm for the sensing element with E-beam evaporator; (3) deposition of 9 μm Parylene C with Parylene vacuum coating system (PDS, Specialty Coating System, Inc., IN), (4) deposition and patterning of a metal layer of Cr/Au for electrode leads (2 μm) with E-beam evaporator, (5) deposition and patterning of another thick layer of Parylene C (12 μm) to form the device structure, and (6) etching the underneath silicon sacrificial layer with XeF₂ etching system leading to the final device. The resulting sensor bodys were 4 cm in length, 320 μm in width and 21 μm in thickness. The fabrication process illustrates the application of Ti and Pt as the heating and sensing element. The Ti/Pt sensing elements (Strip of 280 μm in length by 2 μm in width) were encapsulated with parylene which was in direct contact with the blood flow. They offer low resistance drift, large range of thermal stability, low 1/f noise with absence of piezoresistive effect, and resistance to corrosion/oxidation.

### Example 2

The following example shows one method of using a biosensor of the present disclosure.

The sensors were integrated to an electrical coaxial wire as guide wire catheter application for intravascular shear stress analysis. The Cr/Au electrode leads were connected to an electrical coaxial wire (Precision Interconnet, Portland, Oreg.) using the biocompatible conductive epoxy (H20E, www.epotek.com) that was cured at 90° C. over 3 hours. The electrical coaxial wire allowed for transmitting the electrical signals from the arterial circulation to the external circuitry. The sensor was mounted to the coaxial wire at 4 cm from the tip analogous to the entrance length required to deliver well defined laminar flow field. This distance avoided flow disturbance at the tip of the coaxial wire. The biocompatible epoxy anchored the sensing elements on the coaxial wire surface. The coaxial wire was 0.4 mm in diameter, and the sensing element was 80 μm in width and 240 μm in length.

Using the fluoroscope in the animal angiographic lab, the operator was able to visualize and steer the sensor wire in the aorta of the New Zealand White rabbits to the anatomic regions of interest; namely, aortic arch and abdominal aorta. Contrast dye was injected to delineate the position of the wire in relation to the inner diameter of the aorta.

### Calibration of the Polymer Sensors

Based on the heat transfer principle, the voltage output of the MEMS sensors under the constant current detection circuits was sensitive to the fluctuation in ambient temperature. The temperature overheat ratio (α_{T}) is defined as temperature variations of the sensor over the ambient temperature (T₀):

$${α}_{T} = \frac{( T - {T}_{0} )}{{T}_{0}} \qquad ( 2 )$$

where T denotes the temperature of the sensor. The relation between resistance and temperature overheat ratios is expressed as:

$${α}_{R} = \frac{( R - {R}_{0} )}{R} = α ( T - {T}_{0} ) \qquad ( 3 )$$

where α is temperature coefficient of resistance or TCR. For shear stress measurement, a high overheat ratio is applied by passing higher current and by generating a “hot” sensing element to stabilize the sensor. Calibration was performed in a 2-D flow channel for individual sensors to establish a relationship between heat exchange (from the heated sensing element to the flow field) and shear stress over a range of steady flow rates (Qₙ) in the presence of rabbit blood flow at 37.8° C. For a Newtonian fluid and at steady state, the theoretical shear stress value in a 2-D flow channel was calculated using the following:

$${τ}_{w} = \frac{6 {Q}_{n} μ}{{h}^{2} w} \qquad ( 4 )$$

where τ_{w} is the wall shear stress, μ is the blood viscosity, and h and w are the dimensions of the flow channel. The viscosity of the blood as a function of flow rate was obtained using a viscometer (Brookfield, Middleboro, Mass.). The individually calibrated sensors were then deployed to the NZW rabbit's aorta for real-time shear stress assessment.

### In Vivo Assessment of Intravascular Shear Stress

Real-time shear stress measurements from the NZW rabbit's aorta was acquired; specifically, abdominal aorta and aortic arch. Deployment of the polymer device into the rabbit's aorta was performed in compliance with the Institutional Animal Care and Use Committee in the Heart Institute of the Good Samaritan Hospital, Los Angeles, which is accredited by the American Association for Accreditation for Laboratory Animal Care.

Five male New Zealand White (NZW) rabbits (10 to 12 weeks, mean body weight 2,105±47 g) were acquired from a local breeder (Irish Farms, Norco, Calif.) and maintained by the USC vivaria in accordance with the National Institutes of Health guidelines. After a 7-day quarantine period, the rabbits were anesthetized for percutaneous access according to the institutional review committee, and anesthesia were induced with an intramuscular injection of 100 mg/kg ketamine (Fort Dodge Laboratories, Inc) combined with 1 mg/kg Acepromazine (Aveco Co.). A 23 gauge hypodermic needle and a 26 gauge guide wire were introduced into the left femoral artery via a cut-down. A rabbit femoral catheter (0.023″ID×0.038″OD) was passed through the left femoral artery. The circulatory system of the individual animals was heparinized (100 U/kg) prior to sensor deployment. The catheters and needles were rinsed with heparin at 1000units/mL prior to the procedure. Under the fluoroscopic guidance (Phillips BV-22HQ C-arm), the catheter integrated with the micro vascular device was placed at the abdominal aorta above the renal arties for shear stress measurements under fluoroscopy guidance. Periodic blood pressure measurement was obtained with an automated tail cuff (IITC/Life Science Instruments). The shear stress recordings were synchronized with the rabbit's cardiac cycle via ECG (The ECGenie™, Mouse Specifics). After measurement, the catheter was removed and the femoral artery was tied off.

Development of CFD Stimulation

Generation of 3-D Geometries and Meshes

Computational fluid dynamic (CFD) code was developed for non-Newtonian fluid to simulate real-time shear stress in the abdominal aorta and to compare with the experimental measurements. The luminal geometrical model of the rabbit abdominal aorta was constructed and meshed using a specialized pre-processing program GAMBIT (Fluent Inc., Gambit 2.3.16, Lebanon, N.H., USA). The local effects of branching arteries were assumed to be negligible. The meshed models were then imported into the main CFD solver FLUENT (Fluent Inc., Fluent 6.2.16, Lebanon, N.H., USA) for pulsatile flow simulation. The grid was generated by meshing the inlet surface using Pave scheme type to create unstructured mesh, followed by generating a volume mesh using Cooper scheme type to sweep the mesh node patterns that specified the inlet surface as the “source” faces. The model was composed of 174,510 cells which were primarily the wedge elements. For simulation of wall shear stress, boundary layers immediately adjacent to the wall were constructed to generate sufficient information for characterization of the large fluid velocity gradients near the wall. The diameter of the rabbit abdominal aorta, D, which was measured from angiography during sensor deployment, was set at 2.4 mm. The total length was set at 8.27 times of the diameter to provide sufficient entrance length for the flow to develop.

Using Womersley solution, the pulsatile centerline flow velocity information was used to compute a complex Fourier series approximation for the inlet flow rate pulse. The blood flow was simulated by applying the 3-D Navier-Stokes equations. The governing equations, including mass and momentum equations, were solved in FLUENT for laminar, incompressible, non-Newtonian flow. The arterial wall of rabbit abdominal aorta was assumed to be rigid and impermeable.

At the inlet of the abdominal aorta, a physiological flow waveform was introduced. Using Womersley solution, the transient flow rate information was used to compute a complex Fourier series approximation for the pressure gradient pulse. This profile was implemented by the user defined C++ code. The flow outlet was far downstream where traction-free condition was prescribed. With this approach, the velocity profile become a solution to the 3-D Navier-Strokes equations, and was propagated downstream along the aorta. No-slip boundary condition was implemented along the inner walls.

The flow field was initialized by propagating the constant time-averaged inlet velocity profile downstream into the computational domain. The initial pressure was set to zero in the entire domain as were the two cross-stream velocity components. An iterative scheme that marched toward a converged solution was employed by FLUENT. The second order implicit formulation of the solver was applied for the unsteady simulations. Second order-upwind discretization was applied for the governing equations. The pressure-velocity coupling was based on the SIMPLEC technique.

Results

Properties of Polymer Sensors

The resistance of the sensing element was ˜1.0 kOhm, and the temperature coefficient of resistance was measured to be approximately 0.16%/° C. These properties were compatible for in vivo analysis. The relation between the resistance and temperature was linear, suggesting that the thermal coefficient of resistance (TCR) over this temperature range remained constant.

Calibration of the Polymer Sensors

To account for the non-Newtonian properties of the blood flow, 10 ml blood from the NZW rabbits was collected and assessed the dynamic range of viscosity at 37.8° C. in a 2-D flow channel. The blood viscosity decreased exponentially as the shear rates increased. At shear rate greater than 1,000, the viscosity became asymptotic. The sensing element (240×80×0.1□ μm³) was positioned in a PDMS flow channel (1.32 mm high and 3.0 mm wide) for sensor calibration in the presence of rabbit blood flow at 37.8° C. A non-linear relation between heat dissipation from the sensing element to the blood flow filed as a function to shear stress was obtained. When the sensor reaches the thermal balance status, the power equation is:

Pₑ=P_{b}(ΔT)+P_{f}(ΔT,τ)

Where Pₑ is input electrical power, P_{b} is the power keeping in sensor body, ΔT is the sensor's temperature decrease due to flow τ □ is the shear stress. This equation shows that ΔT has direct relationship with τ, which is demonstrated in FIG. 1.

This calibration curve allowed for conversion of voltage signals to shear stress in the abdominal aorta.

In Vivo Assessment of Intravascular Shear Stress

Conversion of Voltage Signals to Shear Stress in the Abdominal Aorta

Shear stress at the abdominal aorta was calculated using a calibration curve. It responded to a heart rate at ˜200 beats/min. The measured shear stress has a peak value of 30 dynes/cm² and a trough value of 5 dynes/cm².

The foregoing is offered primarily for illustrative purposes. The present disclosure is not limited to the above described embodiments, and various variations and modifications may be possible without departing from the scope of the present invention.

1. A biosensor comprising:
a sensing element;
a first and a second metal electrode both of which are in contact with the sensing element;
a biocompatible polymer layer encompassing the first and second electrodes; and
a heating element.

2. The biosensor of claim 1, wherein the biocompatible polymer layer is comprised of at least one from the group of poly p-chloroxylylene, polyamide, polyimide, polyurethane, and epoxide resin.

3. The biosensor of claim 1, wherein the biocompatible polymer layer is comprised of poly-p-chloroxylylene.

4. The biosensor of claim 1, further comprising:
a center signal wire in contact with the first electrode;
an insulating layer encompassing the periphery of the center signal wire;
a metal ground in contact with the second electrode and encompassing the periphery of the insulating layer; and
a biocompatible polymer layer encompassing the periphery of the metal ground.

5. The biosensor of claim 4, wherein the sensing element is attached to the center signal wire with a conductive biocompatible polymer.

6. The biosensor of claim 5, wherein the sensing element is further attached to the metal ground with conductive biocompatible polymer.

7. The biosensor of claim 5, wherein the conductive biocompatible polymer is comprised of conductive epoxy resin.

8. The biosensor of claim 6, wherein the conductive biocompatible polymer is comprised of conductive epoxy resin.

9. A method of manufacturing a biosensor comprising the steps of:
depositing a silicon oxide layer on a substrate;
depositing and patterning a first metal sensor on the silicon oxide layer;
depositing a first plastic resin layer on the metal sensor;
etching at least two through holes in the first plastic resin layer;
depositing a second metal layer on the plastic resin layer such that a portion of the second metal layer contacts the first metal layer and a portion of the second metal layer contacts the plastic resin layer;
depositing a second plastic resin layer over the second metal layer; and
separating the substrate from the silicon oxide layer.

10. The method of manufacturing a biosensor according to claim 9, wherein the substrate is comprised of silicon or silicon and an insulating material.

11. The method of manufacturing a biosensor according to claim 9, wherein the first metal layer is comprised of Pt and Ti.

12. The method of manufacturing a biosensor according to claim 9, wherein the second metal layer is comprised of Au and Cr.

13. The method of manufacturing a biosensor according to claim 9, wherein the metal sensor further comprises a heating element.

14. The method of manufacturing a biosensor according to claim 12, wherein the second metal layer is in direct contact with the first metal layer.

15. The biosensor of claim 1, wherein the sensing element is configured to provide a temperature-dependent resistance.

16. The biosensor of claim 1, wherein the biosensor has a tip and the sensing element is configured to measure temperature at the tip.

17. The biosensor of claim 1, wherein the sensing element allows measurement of a flow rate of bodily fluid when the resistance of the sensing element is calibrated with the flow rate.

18. The biosensor of claim 1, wherein the sensing element allows measurement of a pressure of bodily fluid when the resistance of the sensing element is calibrated with the pressure.

19. The sensing element of claim 1 wherein the sensing element is separate from the heating element.
